(19) United States
(12) Patent Application Publication
(10) Pub. No.: US 2024/0127955 A1
(43) Pub. Date: Apr. 18, 2024

(54) SYSTEM AND METHODS FOR MAPPING CANCER CLASSIFICATIONS

(71) Applicant: Grail, LLC, Menlo Park, CA (US)

(72) Inventors: Zhao Dong, Fremont, CA (US); Kathryn Nelson Kurtzman, San Francisco, CA (US); Joerg Bredno, San Francisco, CA (US); Xiaoji Chen, Newark, CA (US); Amber Yeh, Fremont, CA (US); Ting Ma, Sunnyvale, CA (US)

(21) Appl. No.: 18/487,558

(22) Filed: Oct. 16, 2023

Related U.S. Application Data
(60) Provisional application No. 63/416,841, filed on Oct. 17, 2022.

(51) Int. Cl. G16H 50/20; C12Q 1/6827; G16H 10/60

(52) U.S. Cl. CPC G16H 50/20; C12Q 1/6827; G16H 10/60

(57) ABSTRACT

Systems and methods for mapping cancer classification labels are provided. In a method for generating a cancer label for a cancer patient, the method entails acquiring, from a data source, a cancer classification for a cancer in the cancer patient; extracting, from the cancer classification, primary anatomic site, primary histology, and invasiveness (or behavior) of the cancer; identifying, in a lookup table, an entry having the extracted anatomic site, histology, and invasiveness (or behavior); and retrieving a corresponding cancer label in the entry for the cancer.

### CROSS REFERENCE TO RELATED APPLICATIONS

This application claims the benefit under 35 U.S.C. § 119(e) of the U.S. Provisional Application Ser. No. 63/416,841, filed Oct. 17, 2022, the content of which is hereby incorporated by reference in its entirety.

### BACKGROUND

Cancer classification is critical for cancer diagnosis, treatment, and research. Standardized cancer ontologies are essential for standardized and refined diagnosis of the disease. There are formal classification systems such as the Systematized Nomenclature of Medicine Clinical Terms (SNOMED-CT) and the International Classification of Diseases for Oncology (ICD-O) as well as terminology systems such as National Cancer Institute Thesaurus (NCIt), Unified Medical Language System (UMLS), and Medical Subject Headings (MeSH).

The field of cancer care is evolving rapidly, and classification systems need to evolve in step to accommodate new research data and to support current evidence-based clinical decisions. Cancer classification is one of the fundamental provisions of clinical decision support systems. However, classification systems such as ICD-O and SNOMED-CT were not designed specifically to support the computational needs of such decision support systems or to include specific, common genomic alterations of cancer specimens. Meanwhile, cancer classification systems such as ICD-O and SNOMED-CT are slowly iterative, taking years to adopt newer entities, while struggling to incorporate newly defined tumor entities, in particular rare tumors. Newer cancer classification systems have been proposed or developed over the recent years, such as the Galleri® system developed by Grail Inc. and OncoTree by Memorial Sloan Kettering Cancer Center and collaborators.

### SUMMARY

As described, there exist a large number of cancer classification systems. Each of these systems serves different purposes and target audiences and provides an abundance of information that is valuable for cancer managements. On the other hand, however, there is a need to effectively correlate such diverse systems so that data available from each of them can be integrated for improved data analysis and cancer diagnosis and treatments.

Systems and methods for generating cancer classification labels are provided. In some embodiments, the method entails acquiring, from a data source, a cancer classification for a cancer in the patient; extracting, from the cancer classification, primary anatomic site, primary histology, and invasiveness of the cancer; identifying, in a lookup table, an entry having the extracted anatomic site, primary histologic type, and invasiveness; and retrieving corresponding cancer labels for each tumor of interest.

In some embodiments, the method further comprises extracting a human papillomavirus (HPV) status from the cancer classification, wherein the entry further includes the extracted HPV status. In some embodiments, the method further comprises extracting a hormone receptor (HR) level from the cancer classification, wherein the entry further includes the extracted HR level. In some embodiments, the method further comprises extracting a histopathology grade from the cancer classification, wherein the entry further includes the extracted histopathology grade.

In some embodiments, the anatomic site is selected from C00.0 to C97.9 of the International Classification of Diseases for Oncology (ICD-O) cancer classification system. In some embodiments, the histology is selected from M-8000 to M-9989 of the ICD-O cancer classification system.

In some embodiments, the invasiveness is selected from the group consisting of: 0: benign; 1: uncertain whether benign or malignant; 2: in situ; 3: primary malignant or invasive; 6: malignant secondary; and 9: malignant uncertain whether primary or secondary.

In some embodiments, if the lookup table does not include an entry having the extracted anatomic site, histology, and invasiveness, then the method further comprises adding a new entry with the extracted anatomic site, histology, and invasiveness, and add a new cancer label based on the cancer classification.

In some embodiments, the method further comprises analyzing a biological sample isolated from the cancer patient. In some embodiments, the analysis comprises detecting methylation status of one or more DNA fragments. In some embodiments, the DNA fragments are cell free DNA (cfDNA) fragments.

In some embodiments, the biological sample is selected from the group consisting of blood, plasma, serum, semen, milk, urine, saliva and cerebral spinal fluid.

In some embodiments, the method further comprises correlating the methylation status of the DNA fragments to the cancer label to build a cancer classifier. In some embodiments, the method further comprises refining the cancer labels to enhance correlation between methylation status of DNA fragments to the cancer labels, thereby improving the classification accuracy.

Also provided, in one embodiment, is a system comprising a hardware processor and a non-transitory computer-readable storage medium storing executable instructions that, when executed by the hardware processor, cause the processor to perform steps comprising the method of any of the above embodiments.

### BRIEF DESCRIPTION OF DRAWINGS

FIG. 1A illustrates a lookup table for mapping cancer labels, according to an embodiment.

FIG. 1B illustrates a quality control process for generating cancer labels.

FIG. 2A illustrates a flowchart describing a process of sequencing a fragment of cell-free (cf) DNA to obtain a methylation state vector, according to an embodiment.

FIG. 2B is an illustration of the process of FIG. 2A of sequencing a fragment of cell-free (cf) DNA to obtain a methylation state vector, according to an embodiment.

FIG. 3A is a flowchart describing a process of training a cancer classifier, according to an embodiment.

FIG. 3B illustrates an example generation of feature vectors used for training the cancer classifier, according to an embodiment.

FIG. 4A illustrates a flowchart of devices for sequencing nucleic acid samples according to one embodiment.

FIG. 4B is a block diagram of an analytics system, according to an embodiment.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

Cancer classification has significant implications in the clinical setting as well as in population science and for patient communication. Clinically, cancer classification is key to treatment selection and planning. Successful treatment of a cancer provides support for the same or similar treatment of cancers of the same class. Likewise, prognosis and risk predictions can be made based on classifications. In population science, cancers are classified in order to characterize populations in terms of prevalence of the cancers.

Different classification systems, however, have different ontology, data source, and granularity. Based on organ locations, the National Cancer Institute classifies cancers into more than 200 types. Such an anatomic classification system can be further stratified by histologic type of the cancer, such as esophageal adenocarcinoma.

While the anatomic naming system, as well as the other conventional classification systems, has been used for over a century, there has always been a drive to recognize finer subtypes, and this trend has accelerated dramatically with genomic data and other molecular features (e.g., metabonomic and histopathological data). For instance, breast cancers can be classified as estrogen receptor (ER) positive, progesterone receptor (PR) positive, human epidermal growth factor receptor 2 (HER2) positive, any combination of these, and triple negative. More recently, with gene expression data, breast cancers can further be classified into finer molecular subtypes: luminal A, luminal B, HER2 overexpression, normal like, and basal like.

The strong interest in finer classification systems is understandable, as it has potential benefits. A fine classification system is needed to capture the full spectrum of biological diversity and could lead to a better recognition of specific disease mechanisms. Such can help devise treatment options more accurately matched to the patient's disease.

Such a fine-grained classification system, however, also has potential drawbacks. For instance, the finer classes may not be technically robust and supported by sufficient observations to identify the molecular features that define such a subtype and its clinically relevant characteristics like prognosis and expected treatment responses. Second, many of such finer classes may lack a clear biological meaning. Therefore, such top fine classifications may have little impact on treatment selections while being highly costly to curate, and being confusing to practitioners and patients. By contrast, a robust and reliable classification system should be supported by solid clinical and scientific rationale.

At the other end of the spectrum, such as for healthcare education, less granular classifications are likely more effective. In certain other instances, such as for clinical care, classifications at various different levels of granulation may be desired.

Currently available cancer classification systems, therefore, have been developed according to use cases and targeted audience. Class sizes and definitions are tailored toward generality versus specification requirements. Example cancer classification systems for clinical use include those provided by American Joint Committee on Cancer (AJCC), The Union for International Cancer Control's (UICC), and National Comprehensive Cancer Network (NCCN).

In population science, example cancer classification systems include those provided by The Surveillance, Epidemiology, and End Results program (SEER) of the National Cancer Institute, (NCRAS) and The American Cancer Society (ACS).

Cancer classification systems are also developed for clinical studies and test development. The instant inventors and collaborators have developed a cancer classification system for cancer detection programs that can take as input various different types of histological, pathological and/or molecular features. A “cancer detection program” as used herein generally refers to any clinical application that characterizes cancers using a set of measured biomarkers (e.g., histological, pathological and/or molecular features) and is expected to classify them into one of a number of candidate classes. Given the potentially large number of histological, pathological and/or molecular features and the generally limited patient sample sizes, it is discovered that a highly granular classification system is not suitable for data analysis or to develop devices and systems to detect and classify cancer. In some embodiments, a suitable number of cancer classes in a cancer diagnostics program is lower than 100, or alternatively lower than 90, 80, 70, 60, 50, 40, or 30. In some embodiments, a suitable number of cancer classes in a cancer diagnostics program is greater than 10, or alternatively greater than 20, 30, 40, or 50.

Cancer class labeling can be used for appropriately training the cancer detection programs. A cancer detection program can train a cancer classifier by inputting sets of training samples with their feature vectors into the cancer classifier and adjusting classification parameters so that a function of the classifier accurately relates the training feature vectors to their corresponding class labels. The term “class label” or simply “label,” as usually used in the context of supervised machine learning, refers to a discrete attribute whose value a machine learning approach is designed to predict based on the values of other attributes of samples in a class. The action of “class labeling” or simply “labeling” refers to the assignment of labels to each sample, in particular training samples. It is important to assign labels that accurately and consistently assign for each training sample the label for the cancer classifier to gain meaningful information. However, the clinical and patient-level information for training samples may be derived from different sources, and may be described in source data using different terminology, labeling, and/or classification schemes.

For example, there are many different major cancer classification schemes including systems to support cancer registries, clinical and research studies, such as those proposed or described by WHO ICD-10, American Joint Committee on Cancer (AJCC), American Cancer Society (ACS), Surveillance, Epidemiology, and End Result Program (SEER), National Cancer Registration and Analysis Service (NCRAS), the Cancer Genome Atlas Program (TCGA), the Circulating Cell-Free Genome Atlas Study (CCGA). These classification schemes, however, are not consistent. Mapping the resulting classes assigned to tumors based on multiple varying source databases to the labels used in the cancer detection program is therefore an essential step.

A commonly used cancer classification system is the International Classification of Diseases for Oncology (ICD-O) from the WHO. ICD-O is a multi-axial classification of the anatomic site (topography), histology (morphology), behavior, and grading of neoplasms. The anatomic (topography) axis of ICD-O-3 shares some properties with the ICD-10 classification of malignant neoplasms but provides more details for the anatomic site. The current edition of ICD-O is ICD-O-3.2, released in 2019.

The anatomic (topography) axis of ICD-O-3 uses a CNN.N code, ranging from C00 to C97, to denote the anatomic site. For instance, C00-C14 represents various sites within the head and neck, C15 for the esophagus, C16 for the stomach, C17 for small intestines, C18 for colon, C20 for rectum, C21 for anus, C22 for liver, C23 for gallbladder, C25 for pancreas, C34 for lung and bronchus, C38 for heart and pleura, C40 for bone limb, C42 for blood or bone marrow, C47 for peripheral nerves, C49 for soft tissue including muscle, C50 for breast, C51 for vulva, C53 for cervix, C55 for uterus, C56 for ovary, C57 for fallopian tube, C60 for penis, C61 for prostate, C62 for testis, C64 for kidney, C65 for renal pelvis, C66 for ureter, C67 for bladder, C71 for brain, C72 for spinal cord, C73 for thyroid, C75 for endocrine gland, and C77 for lymph node. Further, the third digit in CNN.N represents a sub-site within the anatomic site. For instance, the 0 in C34.0 represents the main bronchus, the 1 in C34.1 represents the upper lobe or lung, and the 9 in C34.9 represents lung NOS (not otherwise specified). A complete listing of the anatomic sites and their codes in ICD-10 can be found at, e.g., icd.who.int/browse10/2010/en #/C00-C97).

The histology (morphology) axis of ICD 03 provides five-digit codes ranging from M-8000/0 to M-9989/3. The first four digits indicate the specific histological term. The fifth digit after the slash (/) is the behavior code (invasiveness), which can be 0: benign; 1: uncertain whether benign or malignant; 2: in situ; 3: malignant; 6: metastatic; or 9: malignant, uncertain whether primary or metastatic. For instance, 8000/3 represents invasive neoplasm, NOS; 8010/3 represents carcinoma, NOS, and 9590/3 represents lymphoma, NOS.

It is noted that the behavior codes may vary between systems. Behavior code 6 (metastatic) is generally not used for primary cancers, which get a behavior code of 3. In SEER (further described below) or NCRAS, behavior code 6 refers to malignant secondary tumor. Metastatic or Non-primary Sites Cases reported to SEER cannot have a metastatic (/6) behavior code. If the only pathologic specimen is from a metastatic site, then it is proper to code the appropriate histology code and the malignant behavior code (/3). The primary site and its metastatic site(s) have the same histology. The behavior is coded as malignant (/3) when malignant metastasis is present. Metastasis can be regional, nodal, or distant. For instance, adenocarcinoma in situ with lymph nodes positive for malignancy is coded as malignant (/3). When the invasive component cannot be found and there are positive lymph nodes, behavior/3 can be assign based on the positive lymph nodes.

Therefore, in some embodiments, the behavior code (invasiveness) may be selected from 0: benign; 1: uncertain whether benign or malignant; 2: in situ; 3: primary invasive cancer, including primary malignant; 6: malignant secondary; or 9: malignant uncertain whether primary or secondary.

The histology codes in ICD-O-3.2 can be found at International classification of diseases for oncology (ICD-O)—3rd edition, 1st revision. 1. Neoplams—classification. I. World Health Organization. II.ICD-O ISBN 978 92 4 154849 6, ISBN 978 92 4 069212 1 (available at apps.who.int/iris/bitstream/handle/10665/96612/9789241548496_eng.pdf).

Likewise, in the Surveillance, Epidemiology, and End Result Program (SEER), cancers are registered by combination of anatomic site of primary cancer and primary histologic type. SEER anatomic site is a 5-digit code (81 sites that can be mapped to ICD-O-3 anatomic codes). There are more than 3,800 combinations of anatomic site and histologic type in the SEER database exports.

It is critical to faithfully map the source classification to the labels used in the instant cancer detection program. Selection of the labels themselves is also important. The chosen label scheme can reflect how cancer is represented in the set of biomarkers used in the cancer detection program and also how these labels will be used when applying the cancer detection program, for example, to inform a diagnostic work-up or guide management decisions or patient prognosis). For example, if the cancer detection program uses circulating tumor DNA as input sample and a targeted methylation assay to identify biomarkers, the these labels can respect that methylation patterns capture the differentiation/cell type of the cell of origin and the aberrant methylation after malignant transformation. Furthermore, any set of labels can be support a first application while further accommodating future activities that are not envisioned today.

Such requirements present significant challenges to the design of the cancer labels, and their mapping to other cancer classification systems. Simply put, no single keys can be sufficient for the purpose of mapping. In the examples of Table 1A, when only the primary anatomic site or the histology is used, no meaningful mapping can be achieved for a variety of cancer diagnosis that could have very different clinical management. Here, even granular anatomic information like C16.2 Body of stomach can mean any of those cancers in Table 1A. Likewise, in Table 1B, cancer diagnosis defined by histology alone like 8085/3 HPV-associated squamous cell carcinoma would be ambiguous for a wide variety of different primary anatomic sites of the cancer.

TABLE 1A
Multiple labels for cancers of the stomach (histology)

| | Cancer | |
|---|---|---|
| ICD-O-3 Histology | label for a classifier | AJCC cancer type |
| 8140/3 Adenocarcinoma | Upper Gastrointestinal | Stomach |
| 8936/3 GIST | Sarcoma | Gastrointestinal |
| | | Stromal Tumor |
| 9699/3 Marginal zone | Lymphoid neoplasm | Hodgkin and |
| B-cell lymphoma | | Non-Hodgkin |
| (MALT) | | Lymphomas |
| 8041/3 Small cell | High-grade neuro- | Stomach |
| carcinoma | endocrine carcinoma | |
| 8240/3 Carcinoid, NOS | Upper Gastrointestinal | Neuroendocrine |
| | | Tumors of the |
| | | Stomach |

TABLE 1B
Multiple labels for HPV-associated
squamous cell carcinoma (anatomic site)

| | Cancer | |
|---|---|---|
| ICD-O-3 Anatomic | label for a classifier | AJCC cancer type |
| C00-C14 Head & Neck | Head and neck | HPV-Mediated (p16+) |
| | | Oropharyngeal Cancer |
| C53.0-C53.9 Cervix uteri | Cervix | Cervix Uteri |
| C21.0-C21.8 Anus | Anus | Anus |
| C20.9 Rectum | Anus | Colon and Rectum |
| C60.1-C60.9 Penis | other/missing | Penis |

Accordingly, a solution is provided for effectively and correctly mapping cancer labels between different classification systems. In one embodiment, the combination of (A) primary anatomic site, (B) histologic type (or morphology), and (C) invasiveness of the cancer (behavior) is used as a key to map the labels.

In addition, for future readiness, the mapping also takes into consideration (D) the status of human papillomavirus (HPV) or Epstein-Barr virus (EBV), (in particular for head & neck cancers) or a hepatitis virus for liver cancers, (note: a viral “status” as used herein refers to whether tumor cells from a patient sample contain DNA of the respective viral origin. Such a viral status can be used as a biomarker for tumor level), (E) hormone receptor (HR) status like estrogen, progesterone, and androgen receptor overexpression for breast and prostate cancers, and/or (F) histopathology grade.

The mapping mechanism can expand beyond existing codes to include study- or application-specific internal nomenclature, for example to represent unique individual cases and samples, or to capture the result of an expert review to curate a label in the presence of uncertainty. For instance, for Lookup ID 1374, the histologic type of the tumor is unknown, an internally designed code “0000” captured such histologic types.

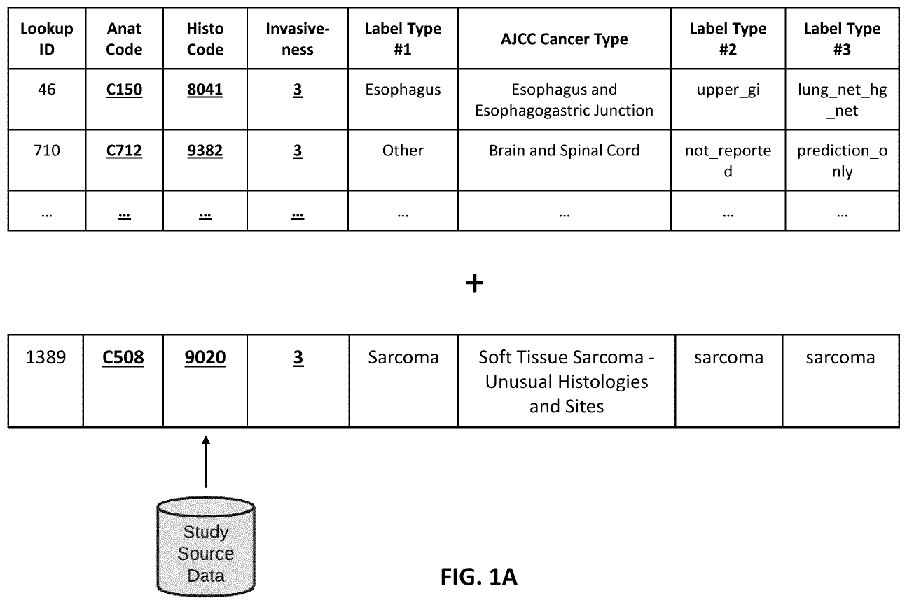

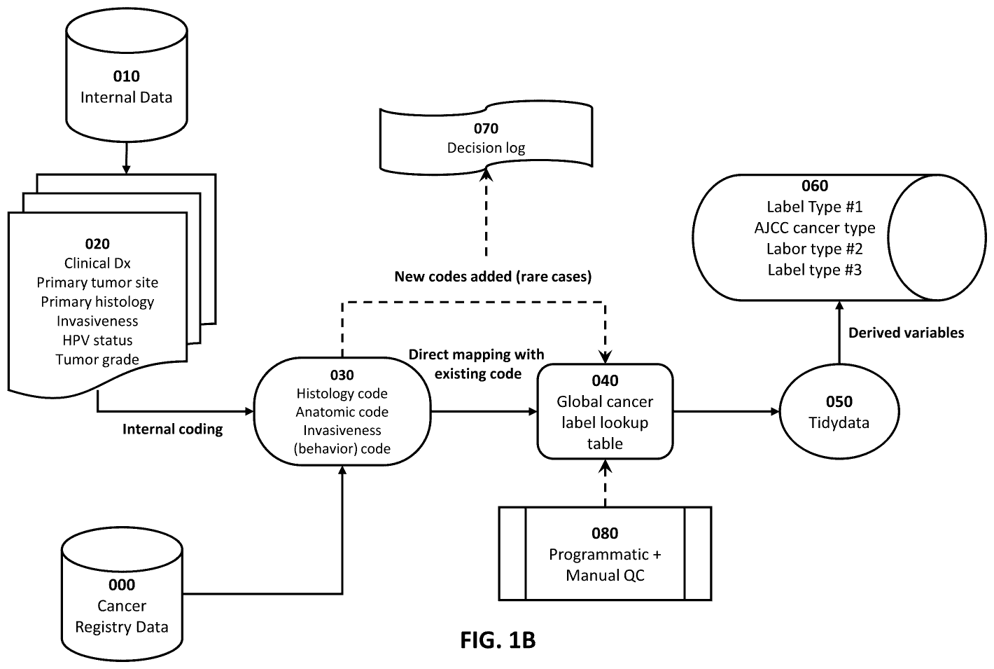

The mapping can be assisted with a lookup table, which includes the mapping keys and output labels to be used in the cancer detection program. The lookup table can also serve as logic for programmatic assignment. An example lookup table is shown in FIG. 1A. As shown in the upper panel, in each entry (row), there is a unique combination of anatomic site (2^{nd} column, “Anat Code”), histologic type (3^{rd} column, “Histo Code”), and behavior/invasiveness (4^{th} column, “Invasiveness”). The unique number (“Lookup ID”) shown in the 1^{st} column is a numerical indicator of this unique combination of keys. An example lookup table is labeled as 040 in FIG. 1B which illustrates a quality control process for the generation of cancer labels.

Each of such entries then corresponds to one or more “outputs” which are cancer labels used in the cancer detection program (e.g., for training purposes). The output can have one or more versions, four of which are illustrated in FIG. 1A (see also 060 in FIG. 1B). In the first type of cancer label (“Label Type #1), for Lookup ID 46, the output cancer label is “Esophagus”, meaning esophageal cancer of any type. In the “AJCC Cancer Type” column, a AJCC style cancer label is the output, such as “Esophagus and Esophagogastric Junction.” The last two columns show some alternative labeling schemes. The AJCC cancer type, a known cancer classification scheme (see, e.g., Amin M B et al., The Eighth Edition AJCC Cancer Staging Manual: Continuing to build a bridge from a population-based to a more “personalized” approach to cancer staging. CA Cancer J Clin. 2017 March; 67(2):93-99), can be included to show that an application- or product-specific cancer label can be assigned together with a label to characterize a sample and case with a previously established labeling scheme.

Also importantly, the lookup table includes entries without existing, corresponding labels in the cancer detection program. As illustrated in the lower panel of FIG. 1A, it is assumed that a combination of anatomic site C508, histology 9020 and behavior 3 did not previously have a corresponding label. Accordingly, new labels can be assigned whenever new cases are added to the training population for a classifier. A new label can be assigned based on the source database (e.g., “sarcoma”) or based on rules that were established to label previous rows and cases in this lookup table (040 in FIG. 1B). In some embodiments, whether the cases actually rise to the level of a new label can be decided by an expert, and the decision of adding or not adding it to the lookup table is logged in a decision log (070). Such accommodation allows easy update of the lookup table and the cancer label scheme used for the cancer detection program.

As explained above, in addition to these three keys, others (e.g., viral status like HPV status, hormone receptor status HR, and/or histopathology grade) can optionally be added to the lookup table as well as a new column(s) (not shown in FIG. 1A).

The mapping is useful for training cancer classifiers, which can then be used for cancer detection and subtyping. The cancer classifier takes patient data with known cancer classification as the training data. The keys (primary anatomic site, primary histology, and invasiveness) are extracted (as 030 in FIG. 1B) from the known cancer classification from the data source, and are compared to the lookup table, so that the source classification is mapped to the output cancer label used in the classifier, which can be saved in a database (e.g., 050 of FIG. 1B) and/or suitably used to train the classifier.

A “data source” as used herein, refers to any collection of information that relates to cancer samples and/or cancer classification. As illustrated in FIG. 1B, a data source may be internal data (010) that include clinical diagnosis of patients and related reports of primary histology etc (020). A data source may also be a public cancer registry (000) that includes suitable clinical diagnosis of patient samples. The information may be stored on a public or private server, or any transitory or non-transitory media as far as it can be accessed by the instant program.

In accordance with one embodiment of the present disclosure, a method for generating a cancer label for a cancer patient is provided. In some embodiments, one or more steps of the method is carried out on a computer that is suitably programmed to carry out one or more of the steps. Certain steps, such as the creation of the lookup table or the quality control thereof, however, can be carried out by a computer program, or alternatively manually (see, e.g., 080 in FIG. 1B). In some embodiments, the method entails acquiring, from a data source, a cancer classification for a cancer in the cancer patient; extracting, from the cancer classification, anatomic site, histology, and invasiveness of the cancer; identifying, in a lookup table, an entry having the extracted anatomic site, histology, and invasiveness; and retrieving a corresponding cancer label in the entry for the cancer.

Typically, clinical samples obtained from actual patients are used for building a cancer classifier. The clinical samples are typically anonymized to protect patient privacy, and each sample has sufficient classification for the cancer the patient suffers(ed). Depending on the source of the patient sample, the cancer classification in the source data can be drastically different. Typical cancer classifications, however, include anatomic site, histology, and invasiveness of the cancer, which are extracted from the patient information.

The anatomic site may be defined according to any data source. Standardized anatomic sites are also provided in various databases, such as ICD-O-3. In some embodiments, the anatomic site is one selected from C00.0 to C97.9 of the International Classification of Diseases for Oncology (ICD-O) cancer classification system (e.g., Table 1).

The histology may be defined according to any data source. Standardized histology characterizations are also provided in various databases, such as ICD-O-03. In some embodiments, the histology is one selected from M-8000 to M-9989 of the ICD-O cancer classification system (e.g., Table 2).

The invasiveness of the cancer is generally classified from benign to metastatic. In some embodiments, the invasiveness is selected from:
0: benign;
1: uncertain whether benign or malignant;
2: in situ;
3: malignant;
6: metastatic; and
9: malignant, uncertain whether primary or metastatic.

Alternatively, in some embodiments, the invasiveness of the cancer can be selected from:
0: benign;
1: uncertain whether benign or malignant;
2: in situ;
3: primary malignant or invasive;
6: malignant secondary; and
9: malignant uncertain whether primary or secondary.

Once extracted, the anatomic site, histologic type, and invasiveness can then be compared to a lookup table (as illustrated in FIG. 1A) to identify a matching entry. The entry also includes cancer labels that can be suitably used in the cancer classifier/detection program of the instant technology.

In some embodiments, from the patient information, additional classification information, such as human papillomavirus (HPV) status, hormone receptor (HR) level and/or histopathology grade can also be extracted.

The human papillomaviruses (HPVs) are a group of more than 150 related viruses. The most common types are found on the skin and appear as warts seen on the hand. There are at least 40 HPV types that can affect the genital areas. Some of these are low-risk and cause genital warts while high-risk types can cause cervical or other types of genital cancer. The high-risk HPV types may also cause head and neck cancer, also called oropharyngeal cancer. A previous HPV infection can result in a reverse transcription of the viral genome into a cell's DNA. Cells with thus modified DNA may be more susceptible to certain cancer types.

In one embodiment, the HPV status is the presence or absence of incorporated viral DNA in the DNA of cancerous cells. In another embodiment, the HPV status includes the type of the HPV.

Hormone receptor status (HR), especially tumor cell overexpression of estrogen receptor, progesteron receptor, or androgen receptor can be useful indicators for the diagnosis or prognosis of certain cancers such as breast cancer and prostate cancer. Example HR status for breast cancer include:
Estrogen receptor overexpression only (“ER-positive” or “ER+”)
Progesterone receptor overexpression only (“PR-positive,” or “PR+”)
Both estrogen and progesterone receptors overexpression (“hormone-responsive”) and
Neither estrogen or progesterone receptor overexpression (“hormone negative” or “HR-” 1).

In some cancer classification, a histopathology grade is also included. There are different systems for such grading, and a non-limiting example is:
Grade X: Grade cannot be assessed (undetermined grade)
Grade 1: Well differentiated (low grade)
Grade 2: Moderately differentiated (intermediate grade)
Grade 3: Poorly differentiated (high grade) or
Grade 4: Undifferentiated (high grade).

Such histopathology grades can also be cancer-type specific, like the Nottingham score or breast cancer or the Gleason grading scheme for prostate cancer.

In some embodiments, when the combination of the above-mentioned keys (anatomic site, histology, and invasiveness, or optionally further with HPV status, HR status, and/or histopathology grade) is not found in the lookup table, a new entry can be added. Such an arrangement helps to ensure that the cancer label system used for classifier training is future-proof In addition to these keys (and optionally a new entry ID), the new entry also includes one or more cancer labels. The cancer label may be as simple as the name of the organ (or anatomic site), the histology, or the combination thereof.

The samples, along with such identified cancer labels can then be used to train a cancer classifier useful for cancer detection, each of which is further described below. In some embodiments, the cancer labels can be further refined based on the performance of the cancer detection program. In some embodiments, various changes can be made to the cancer labels, and the performance is evaluated with respect to correlation between methylation status of DNA fragments and the cancer labels. Revised cancer labels that enhance such correlation can be implemented, which may result in improved cancer detection.

### Example Cancer Classifiers

The instantly disclosed technology for generating cancer labels and using them to map different cancer classifications provides significant value to cancer classifiers which can be used for cancer detection. As described, a cancer classifier characterizes cancers using a set of measured biomarkers. Examples of biomarkers include, but are not limited to, histological, pathological and molecular features. Examples of molecular features include, without limitation, protein expression, genetic mutations, and DNA methylation.

In some embodiments, the cancer classifier uses DNA methylation information, in particular methylation of cell free DNA (cfDNA). Methylation typically occurs in deoxyribonucleic acid (DNA) when a hydrogen atom on the pyrimidine ring of a cytosine base is converted to a methyl group, forming 5-methylcytosine. In particular, methylation tends to occur at dinucleotides of cytosine and guanine referred to herein as “CpG sites”. In other instances, methylation may occur at a cytosine not part of a CpG site or at another nucleotide that is not cytosine; however, these are rarer occurrences. In this present disclosure, methylation is discussed in reference to CpG sites for the sake of clarity. Each CpG site may be methylated or unmethylated.

Identification of anomalously methylated fragments, in comparison to healthy individuals, may provide insight into a subject's cancer status. It is appreciated that an individual can be any animal that may have cancer. An individual may be a rodent, such as a mouse, rat, guinea pig, or a rabbit; a bird, such as a turkey, hen, chicken or other broilers; a farm animal, such as a cow, a horse, a pig, piglet or other free going farm animals. In some embodiments, an individual is a mammal. In some embodiments, an individual is a human.

As is well known in the art, DNA methylation anomalies (compared to healthy controls) can cause different effects, which may contribute to cancer. Anomalous DNA methylation can be identified as hypermethylation or hypomethylation, both of which may be indicative of cancer status. Throughout this disclosure, hypermethylation and hypomethylation is characterized for a DNA fragment, if the DNA fragment comprises more than a threshold number of CpG sites with more than a threshold percentage of those CpG sites being methylated or unmethylated. In accordance with the present description, cfDNA fragments from an individual are treated, for example by converting unmethylated cytosines to uracils, sequenced and the sequence reads compared to a reference genome to identify the methylation states at specific CpG sites within the DNA fragments.

Various challenges arise in the identification of anomalously methylated cfDNA fragments. First off, determining a DNA fragment to be anomalously methylated only holds weight in comparison with a group of control individuals, such that if the control group is small in number, the determination loses confidence due to statistical variability within the smaller size of the control group. Additionally, among a group of control individuals, methylation status can vary which can be difficult to account for when determining a subject's DNA fragments to be anomalously methylated. On another note, methylation of a cytosine at a CpG site causally influences methylation at a subsequent CpG site. To encapsulate this dependency is another challenge in itself.

Those of skill in the art will appreciate that the principles described herein are equally applicable for the detection of methylation in a non-CpG context, including non-cytosine methylation. In such embodiments, the wet laboratory assay used to detect methylation may vary from those described herein. Further, the methylation state vectors discussed herein may contain elements that are generally sites where methylation has or has not occurred (even if those sites are not CpG sites specifically). With that substitution, the remainder of the processes described herein are the same, and consequently the inventive concepts described herein are applicable to those other forms of methylation.

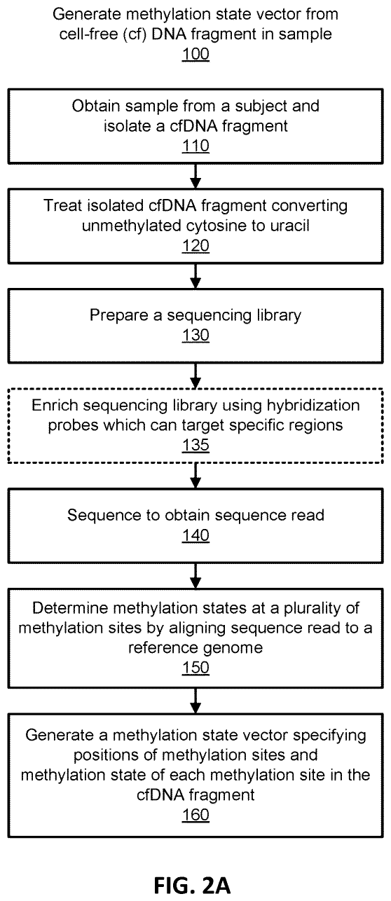

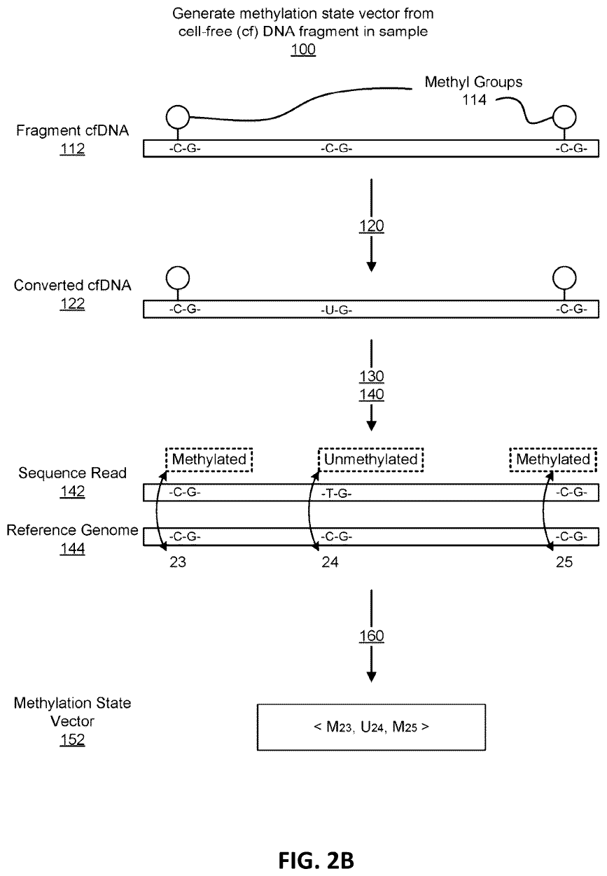

FIG. 2A is a flowchart describing a process 100 of sequencing a fragment of cell-free (cf) DNA to obtain a methylation state vector, according to an embodiment. In step 110, in order to analyze DNA methylation, an analytics system first obtains a sample from an individual comprising a plurality of cfDNA molecules. Generally, samples may be from healthy individuals, subjects known to have or suspected of having cancer, or subjects where no prior information is known. The test sample may be a sample selected from the group consisting of blood, plasma, serum, urine, fecal, and saliva samples. Alternatively, the test sample may be whole blood, a blood fraction (e.g., white blood cells (WBCs)), plasma, serum, semen, milk, urine, saliva, a tissue biopsy, pleural fluid, pericardial fluid, cerebral spinal fluid, and peritoneal fluid. In additional embodiments, the process 100 may be applied to sequence other types of DNA molecules.

From the sample, the analytics system isolates each cfDNA molecule. In step 120, the cfDNA molecules are treated to convert unmethylated cytosines to uracils. In one embodiment, the method uses a bisulfite treatment of the DNA which converts the unmethylated cytosines to uracils without converting the methylated cytosines. For example, a commercial kit such as the EZ DNA Methylation™—Gold, EZ DNA Methylation™—Direct or an EZ DNA Methylation™ —Lightning kit (available from Zymo Research Corp (Irvine, CA)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, MA).

From the converted cfDNA molecules, a sequencing library is prepared in step 130. Optionally, in step 135, the sequencing library may be enriched for cfDNA molecules, or genomic regions, that are informative for cancer status using a plurality of hybridization probes. The hybridization probes are short oligonucleotides capable of hybridizing to particularly specified cfDNA molecules, or targeted regions, and enriching for those fragments or regions for subsequent sequencing and analysis. Hybridization probes may be used to perform a targeted, high-depth analysis of a set of specified CpG sites of interest to the researcher. In one embodiment, the hybridization probes are designed to enrich for DNA molecules that have been treated (e.g., using bisulfite) for conversion of unmethylated cytosines to uracils. Once prepared, the sequencing library or a portion thereof can be sequenced to obtain a plurality of sequence reads, in step 140. The sequence reads may be in a computer-readable, digital format for processing and interpretation by computer software.

From the sequence reads, in step 150, the analytics system determines a location and methylation state for each CpG site based on alignment to a reference genome. In step 160, the analytics system generates a methylation state vector for each fragment specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (e.g., denoted as I). Observed states are states of methylated and unmethylated; whereas, an unobserved state is indeterminate. Indeterminate methylation states may originate from sequencing errors and/or disagreements between methylation states of a DNA fragment's complementary strands. The methylation state vectors may be stored in temporary or persistent computer memory for later use and processing. Further, the analytics system may remove duplicate reads or duplicate methylation state vectors from a single sample. The analytics system may determine that a certain fragment with one or more CpG sites has an indeterminate methylation status over a threshold number or percentage, and may exclude such fragments or selectively include such fragments but build a model accounting for such indeterminate methylation statuses.

FIG. 2B is an illustration of the process 100 of FIG. 2A of sequencing a cfDNA molecule to obtain a methylation state vector, according to an embodiment. As an example, the analytics system receives a cfDNA molecule 112 that, in this example, contains three CpG sites. As shown, the first and third CpG sites of the cfDNA molecule 112 are methylated 114. During the treatment step 120, the cfDNA molecule 112 is converted to generate a converted cfDNA molecule 122. During the treatment 120, the second CpG site which was unmethylated has its cytosine converted to uracil. However, the first and third CpG sites were not converted.

After conversion, a sequencing library 130 is prepared and sequenced 140 generating a sequence read 142. The analytics system aligns 150 the sequence read 142 to a reference genome 144. The reference genome 144 provides the context as to what position in a human genome the fragment cfDNA originates from. In this simplified example, the analytics system aligns 150 the sequence read 142 such that the three CpG sites correlate to CpG sites 23, 24, and 25 (arbitrary reference identifiers used for convenience of description). The analytics system thus generates information both on methylation status of all CpG sites on the cfDNA molecule 112 and the position in the human genome that the CpG sites map to. As shown, the CpG sites on sequence read 142 which were methylated are read as cytosines. In this example, the cytosines appear in the sequence read 142 only in the first and third CpG site which allows one to infer that the first and third CpG sites in the original cfDNA molecule were methylated. Whereas, the second CpG site is read as a thymine (U is converted to T during the sequencing process), and thus, one can infer that the second CpG site was unmethylated in the original cfDNA molecule. With these two pieces of information, the methylation status and location, the analytics system generates 160 a methylation state vector 152 for the fragment cfDNA 112. In this example, the resulting methylation state vector 152 is <M₂₃, U₂₄, M₂₅>, wherein M corresponds to a methylated CpG site, U corresponds to an unmethylated CpG site, and the subscript number corresponds to a position of each CpG site in the reference genome.

The analytics system determines anomalous fragments for a sample using the sample's methylation state vectors, comparing it with methylation state vectors from a control group. For each fragment in a sample, the analytics system determines whether the fragment is an anomalous fragment using the methylation state vector corresponding to the fragment. In one embodiment, the analytics system calculates a p-value score for each methylation state vector describing a probability of observing that methylation state vector or other methylation state vectors even less probable in the healthy control group. The analytics system may determine fragments with a methylation state vector having below a threshold p-value score as anomalous fragments. In another embodiment, the analytics system further labels fragments with at least some number of CpG sites that have over some threshold percentage of methylation or unmethylation as hypermethylated and hypomethylated fragments, respectively. A hypermethylated fragment or a hypomethylated fragment may also be referred to as an unusual fragment with extreme methylation (UFXM). In other embodiments, the analytics system may implement various other probabilistic models for determining anomalous fragments. Examples of other probabilistic models include a mixture model, a deep probabilistic model, etc. In some embodiments, the analytics system may use any combination of the processes described below for identifying anomalous fragments. With the identified anomalous fragments, the analytics system may filter the set of methylation state vectors for a sample for use in other processes, e.g., for use in training and deploying a cancer classifier.

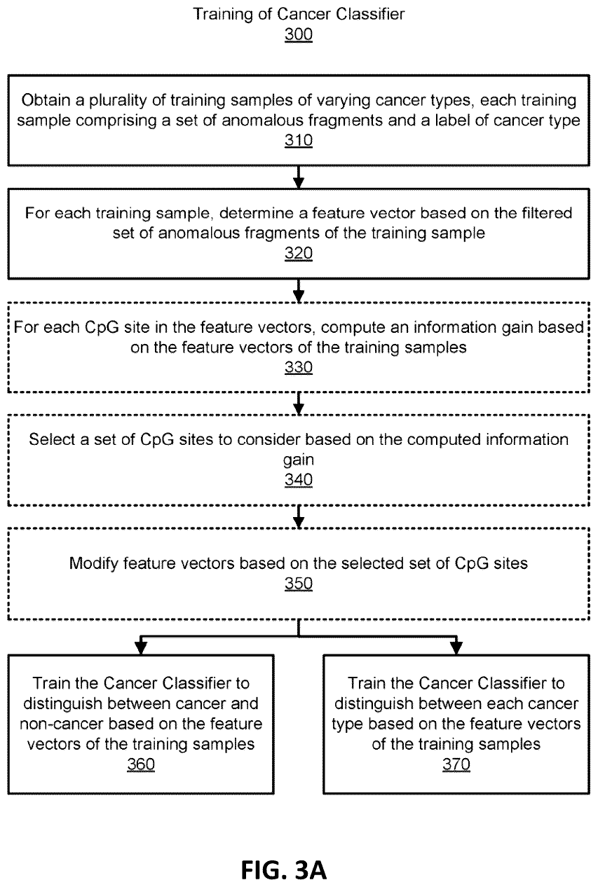

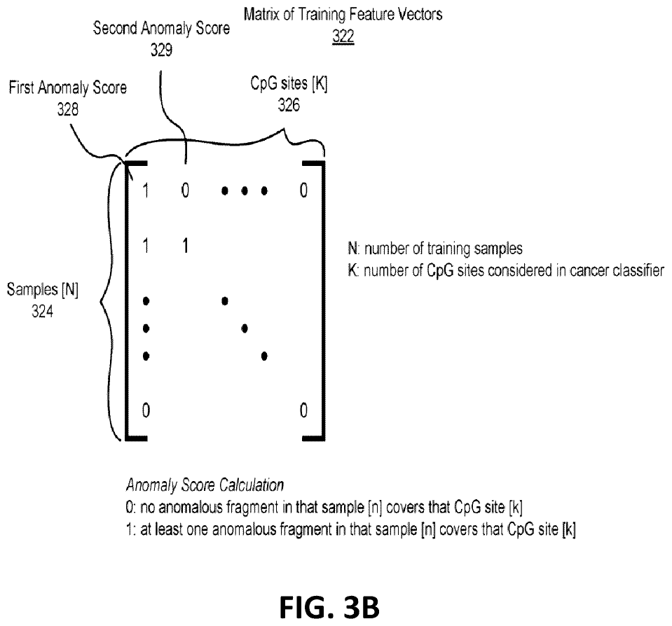

FIG. 3A is a flowchart of devices for sequencing nucleic acid samples according to one embodiment. This illustrative flowchart includes devices such as a sequencer 420 and an analytics system 400. The sequencer 420 and the analytics system 400 may work in tandem to perform one or more steps in the processes described herein.

In various embodiments, the sequencer 420 receives an enriched nucleic acid sample 410. As shown in FIG. 3A, the sequencer 420 can include a graphical user interface 425 that enables user interactions with particular tasks (e.g., initiate sequencing or terminate sequencing) as well as one more loading stations 430 for loading a sequencing cartridge including the enriched fragment samples and/or for loading buffers for performing the sequencing assays. Therefore, once a user of the sequencer 420 has provided the reagents and sequencing cartridge to the loading station 430 of the sequencer 420, the user can initiate sequencing by interacting with the graphical user interface 425 of the sequencer 420. Once initiated, the sequencer 420 performs the sequencing and outputs the sequence reads of the enriched fragments from the nucleic acid sample 410.

In some embodiments, the sequencer 420 is communicatively coupled with the analytics system 400. The analytics system 400 includes some number of computing devices used for processing the sequence reads for various applications such as assessing methylation status at one or more CpG sites, variant calling or quality control. The sequencer 420 may provide the sequence reads in a BAM file format to the analytics system 400. The analytics system 400 can be communicatively coupled to the sequencer 420 through a wireless, wired, or a combination of wireless and wired communication technologies. Generally, the analytics system 400 is configured with a processor and non-transitory computer-readable storage medium storing computer instructions that, when executed by the processor, cause the processor to process the sequence reads or to perform one or more steps of any of the methods or processes disclosed herein.

Referring now to FIG. 3B, FIG. 3B is a block diagram of an analytics system 400 for processing DNA samples according to one embodiment. The analytics system implements one or more computing devices for use in analyzing DNA samples. The analytics system 400 includes a sequence processor 440, sequence database 445, model database 455, models 450, parameter database 465, and score engine 460. In some embodiments, the analytics system 400 performs some or all of the processes 100 of FIG. 2A.

The sequence processor 440 generates methylation state vectors for fragments from a sample. At each CpG site on a fragment, the sequence processor 440 generates a methylation state vector for each fragment specifying a location of the fragment in the reference genome, a number of CpG sites in the fragment, and the methylation state of each CpG site in the fragment whether methylated, unmethylated, or indeterminate via the process 100 of FIG. 2A. The sequence processor 440 may store methylation state vectors for fragments in the sequence database 445. Data in the sequence database 445 may be organized such that the methylation state vectors from a sample are associated to one another.

Further, multiple different models 450 may be stored in the model database 455 or retrieved for use with test samples. In one example, a model is a trained cancer classifier for determining a cancer prediction for a test sample using a feature vector derived from anomalous fragments. The analytics system 400 may train the one or more models 450 and store various trained parameters in the parameter database 465. The analytics system 400 stores the models 450 along with functions in the model database 455.

During inference, the score engine 460 uses the one or more models 450 to return outputs. The score engine 460 accesses the models 450 in the model database 455 along with trained parameters from the parameter database 465. According to each model, the score engine receives an appropriate input for the model and calculates an output based on the received input, the parameters, and a function of each model relating the input and the output. In some use cases, the score engine 460 further calculates metrics correlating to a confidence in the calculated outputs from the model. In other use cases, the score engine 460 calculates other intermediary values for use in the model.

### Training of Cancer Classifier

The cancer classifier may be trained to receive a feature vector for a test sample and determine whether the test sample is from a test subject that has cancer or, more specifically, a particular cancer type. The cancer type (cancer label) can be generated from the mapping method as described above from data sources that come with the test sample. In one embodiment, the cancer label assignment can be adjusted to reflect the features that are available for classification when using methylation of cfDNA as input.

The cancer classifier comprises a plurality of classification parameters and a function representing a relation between the feature vector as input and the cancer prediction as output determined by the function operating on the input feature vector with the classification parameters.

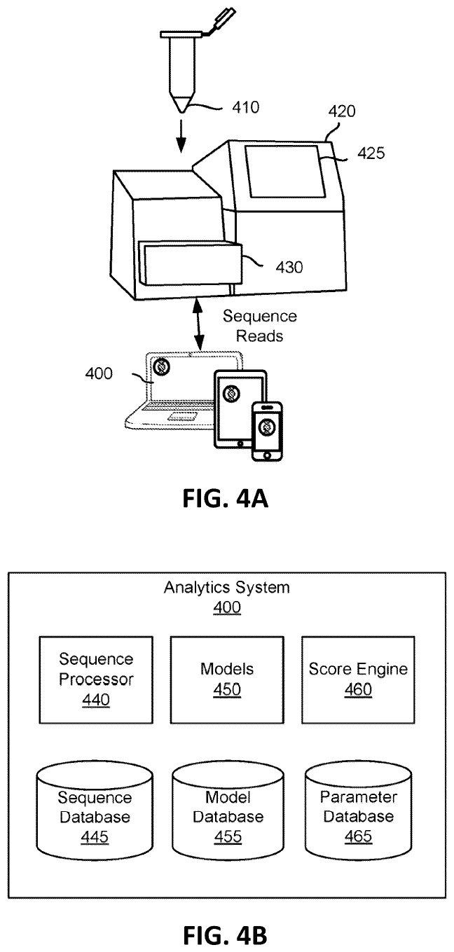

FIG. 4A is a flowchart describing a process 300 of training a cancer classifier, according to an embodiment. In step 310, the analytics system obtains a plurality of training samples each having a set of anomalous fragments. The plurality of training samples includes any combination of samples from healthy individuals with a general label of “non-cancer,” samples from subjects with a general label of “cancer” or a specific label (e.g., “breast cancer,” “lung cancer,” etc.). The training samples from subjects for one cancer type may be termed a cohort for that cancer type or a cancer type cohort.

For each of the training samples, a label may be assigned. In some instances, the training samples may include some type of labels when obtained. After the training samples are obtained, “standardized” labels may be assigned to the training samples. Further, in some embodiments, training labels may be assigned to the training samples, where the training labels are determined based on the standardized label. The standardized labels and training labels are described in more detail below.

In step 320, the analytics system determines, for each training sample, a feature vector based on the set of anomalous fragments of the training sample. The analytics system calculates an anomaly score for each CpG site in an initial set of CpG sites. The initial set of CpG sites may be all CpG sites in the human genome or some portion thereof—which may be on the order of 10⁴, 10⁵, 10⁶, 10⁷, 10⁸, etc. In one embodiment, the analytics system defines the anomaly score for the feature vector with a binary scoring based on whether there is an anomalous fragment in the set of anomalous fragments that encompasses the CpG site. In another embodiment, the analytics system defines the anomaly score based on a count of anomalous fragments overlapping the CpG site. In one example, the analytics system may use a trinary scoring assigning a first score for lack of presence of anomalous fragments, a second score for presence of a few anomalous fragments, and a third score for presence of more than a few anomalous fragments. For example, the analytics system counts 5 anomalous fragment in a sample that overlap the CpG site and calculates an anomaly score based on the count of 5.

Once all anomaly scores are determined for a training sample, the analytics system determines the feature vector as a vector of elements including, for each element, one of the anomaly scores associated with one of the CpG sites in an initial set. The analytics system normalizes the anomaly scores of the feature vector based on a coverage of the sample. Here, coverage refers to a median or average sequencing depth over all CpG sites covered by the initial set of CpG sites used in the classifier, or based on the set of anomalous fragments for a given training sample.

As an example, reference is now made to FIG. 4B illustrating a matrix of training feature vectors 322. In this example, the analytics system has identified CpG sites [K] 326 for consideration in generating feature vectors for the cancer classifier. The analytics system selects training samples [N] 324. The analytics system determines a first anomaly score 328 for a first arbitrary CpG site [k1] to be used in the feature vector for a training sample [n1]. The analytics system checks each anomalous fragment in the set of anomalous fragments. If the analytics system identifies at least one anomalous fragment that includes the first CpG site, then the analytics system determines the first anomaly score 328 for the first CpG site as 1, as illustrated in FIG. 4B. Considering a second arbitrary CpG site [k2], the analytics system similarly checks the set of anomalous fragments for at least one that includes the second CpG site [k2]. If the analytics system does not find any such anomalous fragment that includes the second CpG site, the analytics system determines a second anomaly score 329 for the second CpG site [k2] to be 0, as illustrated in FIG. 4B. Once the analytics system determines all the anomaly scores for the initial set of CpG sites, the analytics system determines the feature vector for the first training sample [n1] including the anomaly scores with the feature vector including the first anomaly score 328 of 1 for the first CpG site [k1] and the second anomaly score 329 of 0 for the second CpG site [k2] and subsequent anomaly scores, thus forming a feature vector [1, 0, . . . ].

The analytics system may further limit the CpG sites considered for use in the cancer classifier, because some CpG sites in the initial set of CpG sites may not be as informative as others in distinguishing between cancer types, or may be duplicative with other CpG sites. In one embodiment, in step 330, the analytics system computes an information gain for each cancer type and for each CpG site in the initial set to determine whether to include that CpG site in the classifier. The information gain is computed for training samples with a given cancer type compared to all other samples. That is, how many bits of information about the cancer type are gained if it is known whether there is an anomalous fragment overlapping a particular CpG site. For a given cancer type, the analytics system uses this information to rank CpG sites based on how cancer specific they are, and in step 340, the ranked CpG sites for each cancer type are greedily added (selected) to a selected set of CpG sites based on their rank for use in the cancer classifier. In additional embodiments, the analytics system may consider other selection criteria for selecting informative CpG sites to be used in the cancer classifier. In one embodiment, in step 350, according to the selected set of CpG sites from the initial set, the analytics system may modify the feature vectors of the training samples. For example, the analytics system may truncate feature vectors to remove anomaly scores corresponding to CpG sites not in the selected set of CpG sites.

With the feature vectors of the training samples, the analytics system may train the cancer classifier in any of a number of ways. The feature vectors may correspond to the initial set of CpG sites from step 320 or to the selected set of CpG sites from step 350. In one embodiment, the analytics system trains 360 a binary cancer classifier to distinguish between cancer and non-cancer based on the feature vectors of the training samples. In this manner, the analytics system uses training samples that include both non-cancer samples from healthy individuals and cancer samples from subjects. Each training sample has one of the two labels “cancer” or “non-cancer.” In this embodiment, the classifier outputs a cancer prediction indicating the likelihood of the presence or absence of cancer.

In one embodiment, the analytics system trains 450 a multiclass cancer classifier to distinguish between many cancer types, based on the tissue of origin (TOO), histologic type or both. Cancer types include one or more cancers and may include a non-cancer type (may also include any additional other diseases or genetic disorders, etc.). To do so, the analytics system uses the cancer type cohorts and may also include or not include a non-cancer type cohort. In this multi-cancer embodiment, the cancer classifier is trained to determine a cancer prediction (or, more specifically, a TOO and/or histologic type prediction) that comprises a prediction value for each of the cancer types being classified for. The prediction values may correspond to a likelihood that a given training sample (and during inference, a test sample) has each of the cancer types. In one implementation, the prediction values are scored between 0 and 100, wherein the cumulation of the prediction values equals 100. For example, the cancer classifier returns a cancer prediction including a prediction value for breast cancer, lung cancer, and non-cancer. For example, the classifier can return a cancer prediction that a test sample is 65% likelihood of breast cancer, 25% likelihood of lung cancer, and 10% likelihood of non-cancer. The analytics system may further evaluate the prediction values to generate a prediction of a presence of one or more cancers in the sample. In some embodiments, the prediction may be referred to as a TOO prediction indicating one or more TOO labels, e.g., a first TOO label with the highest prediction value, a second TOO label with the second highest prediction value, etc. In some embodiments, the prediction may be referred to as a histologic type prediction indicating one or more histologic type labels, e.g., a first histologic type label with the highest prediction value, a second histologic type label with the second highest prediction value, etc. Continuing with the example above and given the percentages, in this example the system may determine that the sample has breast cancer given that breast cancer has the highest likelihood.

In both embodiments, the analytics system trains the cancer classifier by inputting sets of training samples with their feature vectors into the cancer classifier and adjusting classification parameters so that a function of the classifier accurately relates the training feature vectors to their corresponding label. The analytics system may group the training samples into sets of one or more training samples for iterative batch training of the cancer classifier. After inputting all sets of training samples including their training feature vectors and adjusting the classification parameters, the cancer classifier is sufficiently trained to label test samples according to their feature vector within some margin of error. The analytics system may train the cancer classifier according to any one of a number of methods. As an example, the binary cancer classifier may be a L2-regularized logistic regression classifier that is trained using a log-loss function. As another example, the multi-cancer classifier may be a multinomial logistic regression. In practice either type of cancer classifier may be trained using other techniques. These techniques are numerous including potential use of kernel methods, random forest classifier, a mixture model, an autoencoder model, machine learning algorithms such as multilayer neural networks, etc.

### Use of Cancer Classifier

Once the classifier is trained with test samples and their corresponding cancer labels, the classifier can then be used to detect cancer for patients. Data acquisition, such as obtaining cfDNA and detection of DNA methylation, can follow the same procedure as used in the training. Taking such data as input, then the output is the predicted cancer type for the patient.

The predicted cancer class, in some embodiments, is one of the cancer labels used in the training, such as those illustrated in FIG. 1A. In general, these labels are less granular than many other cancer classification systems. In some embodiments, the lookup table can be used in a reverse manner, that is, to identify a listing of all combinations of keys (anatomic site, histology, and invasiveness) all of which correspond to the predicted cancer label. Even though the cancer detection program may not provide the more granular prediction/detection of cancer, it provides meaningful guidance for further examination.

The cancer labels defined following the process in FIG. 1A, in some embodiments, can be adjusted after one iteration of classifier training to improve the ability of a classifier to correctly predict labels. For example, labels might be chosen to reflect the methylation status of cancer cells in different regions of the genome. A second classifier can then be trained with these new labels.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A computer-implemented method for generating a cancer label for a cancer patient, comprising:
acquiring, from a data source, a cancer classification for a cancer in the cancer patient;
extracting, from the cancer classification, anatomic site, histology, and invasiveness of the cancer;
identifying, in a lookup table, an entry having the extracted anatomic site, histology, and invasiveness; and
retrieving a corresponding cancer label in the entry for the cancer.

2. The method of claim 1, further comprising extracting a human papillomavirus (HPV) status from the cancer classification, wherein the entry further includes the extracted HPV status.

3. The method of claim 1, further comprising extracting a hormone receptor (HR) level from the cancer classification, wherein the entry further includes the extracted HR level.

4. The method of claim 1, further comprising extracting a histopathology grade from the cancer classification, wherein the entry further includes the extracted histopathology grade.

5. The method of claim 1, wherein the anatomic site is selected from C00.0 to C97.9 of the International Classification of Diseases for Oncology (ICD-O) cancer classification system.

6. The method of claim 1, wherein the histology is selected from M-8000 to M-9989 of the ICD-O cancer classification system.

7. The method of claim 1, wherein the invasiveness is selected from the group consisting of:
0: benign;
1: uncertain whether benign or malignant;
2: in situ;
3: primary malignant or invasive;
6: malignant secondary; and
9: malignant uncertain whether primary or secondary.

8. The method of claim 1, wherein if the lookup table does not include an entry having the extracted anatomic site, histology, and invasiveness, then the method further comprises adding a new entry with the extracted anatomic site, histology, and invasiveness, and add a new cancer label based on the cancer classification.

9. The method of claim 1, further comprising analyzing a biological sample isolated from the cancer patient.

10. The method of claim 9, wherein the analysis comprises detecting methylation status of one or more DNA fragments.

11. The method of claim 10, wherein the DNA fragments are cell free DNA (cfDNA) fragments.

12. The method of claim 9, wherein the biological sample is selected from the group consisting of blood, plasma, serum, semen, milk, urine, saliva and cerebral spinal fluid.

13. The method of claim 10, further comprising correlating the methylation status of the DNA fragments to the cancer label to build a cancer classifier.

14. The method of claim 10, further comprising refining the cancer labels to enhance correlation between methylation status of DNA fragments to the cancer labels, thereby improving the classification accuracy.

15. A system comprising a hardware processor and a non-transitory computer-readable storage medium storing executable instructions that, when executed by the hardware processor, cause the processor to perform steps comprising the method of claim 1.
